(19) United States
(12) Plant Patent Application Publication
(10) Pub. No.: US 2025/0386752 P1
(43) Pub. Date: Dec. 18, 2025

(54) Echinacea Plant Named 'BullEchipur 121'

(50) ;

(71) Applicant: Hartwig Bull, Gõnnebek (DE)

(72) Inventor: Hartwig Bull, Gõnnebek (DE)

(21) Appl. No.: 19/235,215

(22) Filed: Jun. 11, 2025

Related U.S. Application Data
(60) Provisional application No. 63/731,837, filed on Jun. 12, 2024.

(51) Int. Cl. A01H 6/14; A01H 5/02

(52) U.S. Cl. CPC A01H 6/1448; A01H 5/02
USPC PLT/428

(57) ABSTRACT

A new and distinct variety of Echinacea plant named ‘BullEchipur 121’ having orange colored flowers, good growth habit, more uniformity, good branching plant, and good propagation rates.

### CROSS REFERENCE TO RELATED APPLICATIONS

This application claims the benefit of priority from U.S. Provisional Patent Application No. 63/731,837, filed Jun. 12, 2024, which is incorporated herein by reference in its entirety.

Botanical classification: Echinacea purpurea.

Varietal denomination: ‘BULLECHIPUR 121’.

### BACKGROUND OF THE INVENTION

The present invention relates to a new and distinct cultivar of Echinacea, botanically known as Echinacea purpurea, and hereinafter referred to by the name ‘BullEchipur 121’.

The new Echinacea was discovered by the Inventor in August 2021, in a controlled environment in Gönnebek, Germany, as a part of a planned breeding program. The new Echinacea was selected on the basis of orange colored flowers, good growth habit, more uniformity, good branching plant, and good propagation rates.

Asexual propagation of the new cultivar by in vitro culture since September 2021, in Gönnebek, Germany, has shown that the unique features of this new Echinacea are stable and reproduced true to type in successive generations.

### SUMMARY OF THE INVENTION

The following traits have been repeatedly observed and are determined to be unique characteristics of ‘BullEchipur 121’. These characteristics in combination distinguish ‘BullEchipur 121’ as a new and distinct cultivar:
1. Medium compact growths
2. Vibrant orange flowers
3. Lots of flowers
4. More branching habit
5. Very uniform growth
6. Nice green leaves
7. Fast crop time

In side-by-side comparisons conducted by the Inventor in Gönnebek, Germany, plants of the new Echinacea differed from plants of the female parent, Bull 2019-17 of Echinacea purpurea, in the following characteristics:
1. More vigorous
2. Orange flower color
3. Good branching
4. Bigger plant habit

In side-by-side comparisons conducted by the Inventor in Gönnebek, Germany, plants of the new Echinacea differed from plants of the male parent, Bull 2020-02 of Echinacea purpurea, in the following characteristics:
1. More compact
2. More intensive orange flower color
3. More branches
4. Lots of flowers

Plants of the new Echinacea can be compared to the plants of the Echinacea cultivar ‘Cheyenne Spirit’, believed to be unpatented. In side-by-side comparisons conducted by the Inventor in Gönnebek, Germany, plants of the new Echinacea differed from plants of the cultivar ‘Cheyenne Spirit’ in the following characteristics:
1. More compact
2. Pure orange flowers
3. More flowers
4. More branching habit
5. Very even habit
6. Nice cleaner leaves

### DESCRIPTION OF THE DRAWINGS

The accompanying colored photographs illustrate the overall appearance of the new Echinacea, showing the colors as true as it is reasonably possible to obtain in colored reproductions of this type. Colors in the photographs may differ slightly from the color values cited in the detailed botanical description, which accurately describes the colors of the new Echinacea.

FIG. 1 comprises a top perspective view of a typical plant of ‘BullEchipur 121’ shown in natural lighting.

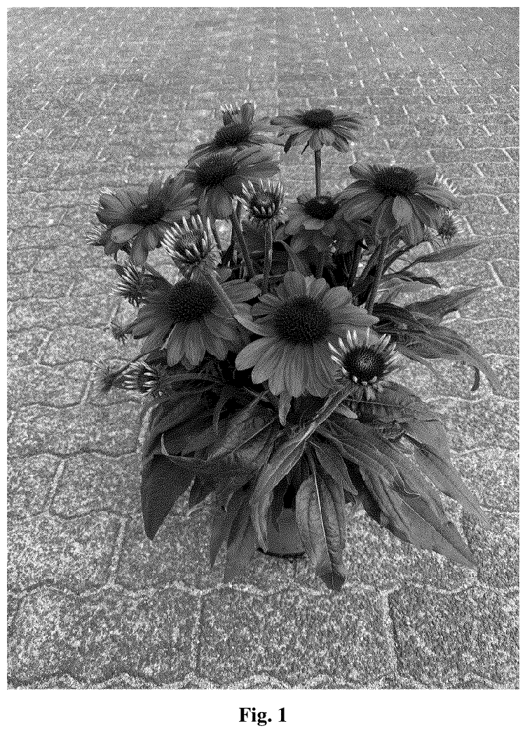

### DESCRIPTION OF THE PLANT

The cultivar Echinacea has not been observed under all possible environmental conditions. The phenotype may vary somewhat with variations in environment such as temperature and light intensity without, however, any variance in genotype.

The aforementioned photographs and following observations and measurements describe plants of the new Echinacea that were grown in an outdoor nursery in Gönnebek, Germany. During the production of the plants, temperatures averaged about 18-20° C. Plants used for the photographs and description were about 22 weeks from planting. Color references are made to The Royal Horticultural Society Colour Chart, 2015 version, except where general terms of ordinary dictionary significance are used.

### PLANT

Botanical classification: Echinacea purpurea.
Parentage: Bull 2019-17 and Bull 2020-02.
Propagation:
   Time to initiate roots.—About 7 days.
   Time to develop roots.—About 40-50 days.
   Time to produce a finished flowering plant.—About 12-14 weeks.
   Root description.—Lots of roots, free-branching, nice, healthy-looking root system.
Plant description:
   Growth rate/vigor.—Compact, upright.
   Plant height.—40-55 cm.
   Plant width.—35-40 cm.
   Branching habit.—Lot of branches, upright, shows a medium compact plant.
   Lateral branches.—Number per plant: 20-25. Length: 2-35 cm. Diameter: 3-8 mm. Angle: 1-5 cm. Strength: Strong. Texture: Pubescent. Color: 146C. Internode length: 1-5 cm.
   Foliage.—Length: 4-25 cm. Width: 1-7 cm. Shape: Lanceolate. Shape of Apex: Acuminate. Shape of Base: Attenuate to Acute. Orientation and aspect: Straight to curved. Texture: Upper surface: Rough. Lower surface: Rough. Young leaves: Upper surface color: 137A. Lower surface color: 137C. Fully expanded leaves: Upper surface color: 137A Lower surface color: 137C. Venation: Type: Cross-venulate to longitudinal. Color: Upper surface: 145A. Lower surface: 145B. Margin: Entire.
Flowers:
   Bud.—Stage of opening: Single type blossom. Shape: Elliptic shaped ray florets and tubular disk florets. Diameter: 1-3.5 cm. Length: 1.2-2.5 cm. Color: N137C.
   Natural flowering season.—13-14 weeks after planting.
   Inflorescence.—Form: Single flower. Disc and ray floret arrangement: Single composite flower. Average number per plant: 20-30. Average number per branch: 1-3. Fragrance: None. Diameter: 4-10 cm. Height: 2-4.5 cm. Diameter of disc: 1.5-4 cm. Receptacle height: 1 cm. Receptacle diameter: 1.5-4 cm. Ray florets: Number per inflorescence: 16-24. Arrangement: In two or three whorls. Length: 1.5-3.5 cm. Width: 0.9-1.5 cm. Shape: Ellipse. Shape of Apex: Obtuse. Shape of Base: Acute. Margin: Entire. Texture: Upper surface: Smooth, glabrous. Lower surface: Rough. Color: When opening: Upper surface: N30A. Lower surface: 35A. At maturity: Upper surface: N30A. Lower surface: 35A. Ground color description: Orange. Disc florets: Number per inflorescence: Numerous, too many to count. Arrangement: In center of receptacle. Length: 8-15 mm. Width: 1-2 mm. Shape: Tubular. Shape of Apex: Sharp. Color: Immature: 181A. At maturity: 181A. Phyllaries: Number per inflorescence: 35-50.Arrangement: In 3-4 whorls. Length: 0.5-1.5 cm. Width: 2-3 mm. Shape: Lanceolate. Shape of Apex: Elongate, oval. Shape of Base: Fused. Margin: Entire. Texture: Upper surface: Rough. Lower surface: Rough. Color: Immature: Upper surface: NN137C. Lower surface: NN137C. At maturity: Upper surface: NN137C. Lower surface: NN137C.
Reproductive organs:
   Androecium.—Presence: On disk florets. Number: One. Filament: Length: 1-2 mm. Color: 183A. Anther: Shape: Oval. Length: 1 mm. Color: 183B. Pollen: Color: 15B. Amount: Too much to quantify.
   Gynoecium.—Presence: In disk and ray florets. Pistil length: 1-2 mm. Stigma: Shape: Two-parted. Color: 183A. Style: Length: 1-2 mm. Color: 183A. Seeds/fruit: Length: 1-2 mm. Color:
Disease/pest resistance:
   Disease resistance.—No additional disease resistance has been observed beyond the typical resistance in Echinacea plants.
   Pest resistance.—No additional pest resistance has been observed beyond the typical resistance in Echinacea plants.
Weather tolerance:
   Temperature tolerance.—No additional temperature resistance has been observed beyond the typical resistance in Echinacea plants.
   Drought tolerance.—No additional drought resistance has been observed beyond the typical resistance in Echinacea plants.

I claim:

1. A new and distinct cultivar of Echinacea plant named ‘BullEchipur 121’, as illustrated and described.
